(19) United States
(12) Patent Application Publication
(10) Pub. No.: US 2023/0148935 A1
(43) Pub. Date: May 18, 2023

(54) Contactless Electrocardiogram Measurement Device

(71) Applicants: Industry-Academic Cooperation Foundation, Yonsei University, Seoul (KR); Korea Advanced Institute of Science and Technology, Daejeon (KR)

(72) Inventors: Shinill Kang, Seoul (KR); Inkyu Park, Daejeon (KR)

(21) Appl. No.: 17/886,592

(22) Filed: Aug. 12, 2022

(30) Foreign Application Priority Data
Aug. 13, 2021 (KR) 10-2021-0107385

(51) Int. Cl. A61B 5/346; A61B 5/00

(52) U.S. Cl. CPC A61B 5/346; A61B 5/6892; A61B 5/7246; A61B 5/4815; A61B 2562/0219

(57) ABSTRACT

Provided is a contactless electrocardiogram measurement device which may perform a high-quality sleep monitoring while improving a sleep quality of an object person. The contactless electrocardiogram measurement device includes a measurement unit disposed between a vibration medium and a support member to measure vibration generated from a body of an object person that is transmitted from the vibration medium, wherein the measurement unit includes a plate-shaped cover portion interposed between the vibration medium and the support member, and a vibration sensor for detecting the vibration generated in the cover portion.

### CROSS-REFERENCE TO RELATED APPLICATION

This application claims priority to Korean Patent Application No. 10-2021-0107385, filed Aug. 13, 2021, the disclosure of which is hereby incorporated by reference in its entirety.

### BACKGROUND OF THE INVENTION

### Field of the Invention

The following disclosure relates to a contactless electrocardiogram measurement device, and more particularly, to a contactless electrocardiogram measurement device which can indirectly measure an electrocardiogram of an object person through a vibration sensor without direct contact with a body of the object person.

### Description of Related Art

Currently, many people are complaining of a symptom such as fatigue or lethargy due to a phenomenon such as reduced sleep time or difficulty falling into deep sleep. In severe cases, this phenomenon may not only cause such a simple symptom, but also cause high blood pressure, obesity or diabetes, or lead to deteriorated health due to occurrence of a disease related to a cardiovascular system, nerves or brain. As a result, technologies are being developed or commercialized in which sleep of an object person is monitored to diagnose a sleep quality of the object person, respiratory status and cardiovascular function during sleep, sleep apnea or the like, and to diagnose and prevent a disease related to the sleep of the object person at an early stage.

A sleep monitor may be mounted on a wearable device, a mobile device or bedding, and measure the brain wave, electrocardiogram, exercise, sleep or the like of the object person. In this case, it is possible to diagnose and predict various sleep safety-related diseases such as sleep apnea, depression, stress, fibrosis, heart failure and arrhythmia through the electrocardiogram measurement. It is also possible to obtain data for distinguishing stable and deep sleep of the object person from unstable sleep of the object person through the electrocardiogram measurement.

Currently, Korean Patent Publication No. 10-2018-0015336 (entitled, “bed cable for electrocardiogram measurement” and published on Feb. 13, 2018) discloses technology of a bed cable for measuring the electrocardiogram for the sleep monitoring. Referring to FIG. 1, the bed cable for electrocardiogram measurement may include: a mat 1 which supports a measurement object person 0; a plurality of measurement electrodes 2 which receive a micro-current generated from the measurement object person 0 through the skin of the measurement object person 0; at least three connection terminals 3 which are positioned on a support surface of the mat 1 on which the measurement object person 0 is supported, and to which a measurement electrodes 2 are connected; at least two connection terminals 4 which are electrically connected to an electrocardiogram system “E”; and a connection circuit 5 which is positioned in the mat 1 and connects the connection terminal 3 and the connection terminal 4 with each other, wherein at least one of the plurality of measurement electrodes 2 provides the electrocardiogram system “E” connected to the connection terminal 4 with the micro-current generated in a process of depolarizing a heart muscle of the measurement object person 0.

The electrocardiogram measurement technology as described above for sleep monitoring, which is currently disclosed, may have low accessibility. The reason is that the technology may be limited to only a specialized facility to provide this service as most of the bedding needs to be made separately so that the electrodes and terminals are embedded therein. In addition, the electrocardiogram measurement using the electrodes may require the electrodes or another device to be attached to the body of the object person, and may thus disturb the sleep of the object person and also affect a measurement result.

### SUMMARY OF THE INVENTION

An exemplary embodiment of the present disclosure is directed to providing a contactless electrocardiogram measurement device which can indirectly measure an electrocardiogram of an object person through a vibration sensor without direct contact with a body of the object person, and measure vibration more precisely while reducing foreign body sensation felt by the object person.

In one general aspect, a contactless electrocardiogram measurement device includes a measurement unit disposed between a vibration medium and a support member to measure vibration generated from a body of an object person that is transmitted from the vibration medium, wherein the measurement unit includes a plate-shaped cover portion interposed between the vibration medium and the support member, and a vibration sensor for detecting the vibration generated in the cover portion, and the vibration sensor is embedded in the vibration medium or the support member.

In addition, the vibration medium and the support member may be a topper and a mattress, respectively.

In addition, the contactless electrocardiogram measurement device may further include a processor fixed on the support member and receiving data measured by the vibration sensor.

In addition, the contactless electrocardiogram measurement device may further include a case into which the vibration sensor is inserted and which has one side coupled to one surface of the cover portion, wherein the case has the one side open.

In addition, the cover portion may include a plate-shaped cover body having a diameter greater than its thickness and a first screw hole passing through both surfaces of the cover body, the case may include a second screw hole disposed in the one side thereof to face the first screw hole, and the cover body and the case may be screwed to each other.

In addition, a seating groove into which a cable is able to be inserted may be disposed in the one side of the case, and the cable may be wired to the vibration sensor.

In addition, the cover portion may have a disk shape, and both the surfaces of the cover portion may be in contact with the vibration medium and the support member, respectively.

In addition, the vibration medium or the support member may have a cavity disposed in its surface in contact with the cover portion to embed the vibration sensor therein, and an area of the cavity may be smaller than an area of the cover portion.

In addition, wherein the case may be disposed at a diameter center of the cover portion.

In addition, the contactless electrocardiogram measurement device may further include a fixing member for fixing the vibration sensor into the case.

In addition, the cover portion may have hardness greater than that of the vibration medium.

In another general aspect, a method for calculating a contactless electrocardiogram signal by using the contactless electrocardiogram measurement device described above includes: calculating a first electrocardiogram signal of an object person by using the contactless electrocardiogram measurement device; measuring a second electrocardiogram signal of the object person by using a contact electrocardiogram including electrodes; analyzing a correlation between the first electrocardiogram signal and the second electrocardiogram signal; and correcting data by reflecting the analyzed correlation in a calculation formula of the first electrocardiogram signal.

### BRIEF DESCRIPTION OF THE DRAWINGS

FIG. 1 is a view showing a contact electrocardiogram measurement device according to the prior art.

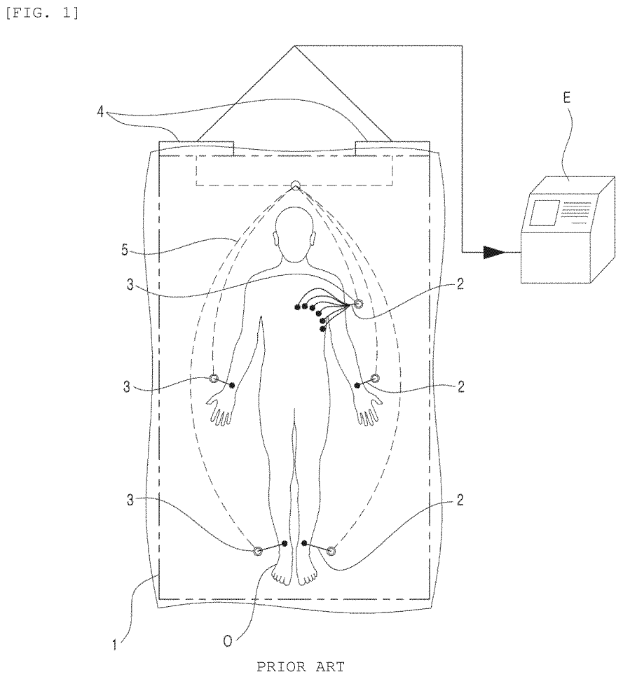

FIG. 2 is a view showing a contactless electrocardiogram measurement device installed on a bed according to the present disclosure.

FIG. 3 is a perspective view showing a measurement unit disposed between a vibration medium and a support member according to the present disclosure.

FIG. 4 is a cross-sectional view showing an example in which the measurement unit is disposed between the vibration medium and the support member according to the present disclosure.

FIG. 5 is a perspective view of the measurement unit according to the present disclosure.

FIG. 6 is an exploded perspective view of the measurement unit according to the present disclosure.

FIGS. 7A to 9B are views each comparing the measurement unit according to the present disclosure and a device having a different shape.

FIG. 10 is a cross-sectional view showing another example in which the measurement unit is disposed between the vibration medium and the support member according to the present disclosure.

FIG. 11 shows a table comparing actual measurement data and calculated data to which a method for calculating a contactless electrocardiogram signal according to the present disclosure is applied with each other.

### DESCRIPTION OF THE INVENTION

Hereinafter, a contactless electrocardiogram measurement device according to the present disclosure is described in detail with reference to the accompanying drawings. The accompanying drawings are only provided by way of example in order to sufficiently transfer the spirit of the present disclosure to those skilled in the art. Therefore, the present disclosure is not limited to the accompanying drawings provided below, and may be implemented in another form. In addition, like reference numerals denote like elements throughout the specification.

Technical terms and scientific terms used in the specification have the general meanings understood by those skilled in the art to which the present disclosure pertains unless otherwise defined, and descriptions for the known function and configuration unnecessarily obscuring the gist of the present disclosure are omitted in the following description and the accompanying drawings.

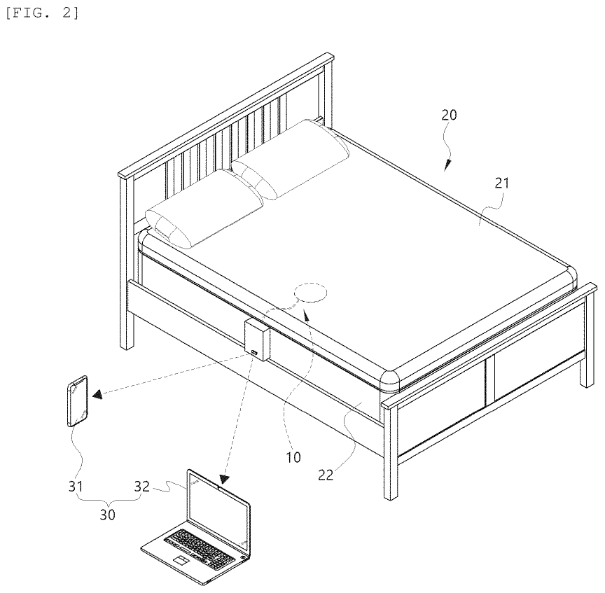

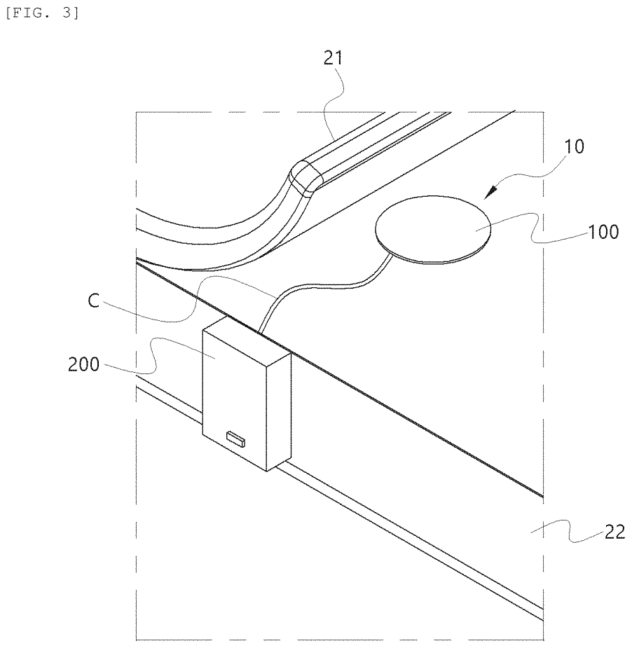

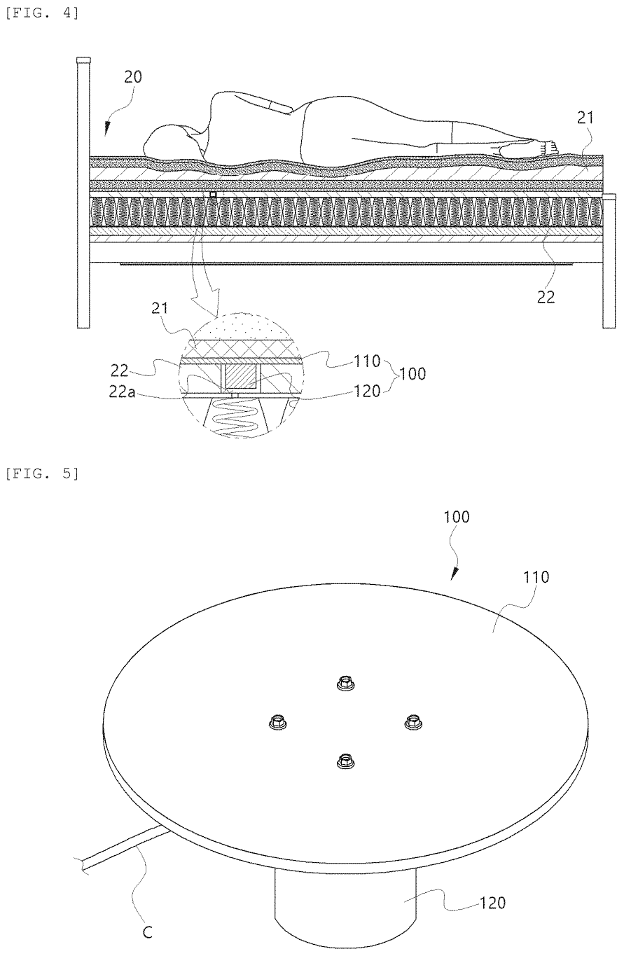

FIGS. 2 to 4 are related to a contactless electrocardiogram measurement device according to the present disclosure: FIG. 2 is a view showing a contactless electrocardiogram measurement device installed on a bed according to the present disclosure; FIG. 3 is a perspective view showing a measurement unit disposed between a vibration medium and a support member according to the present disclosure; and FIG. 4 is a cross-sectional view showing an example in which the measurement unit is disposed between the vibration medium and the support member according to the present disclosure.

Referring to FIG. 2, a contactless electrocardiogram measurement device 10 according to the present disclosure may be installed on a product such as a bed 20 including a vibration medium 21 and a support member 22. In this case, the contactless electrocardiogram measurement device 10 can be installed in any of various product groups such as a vehicle seat and a floor mat when including the vibration medium 21 and the support member 22. In the case of the bed 20, the vibration medium 21 and the support member 22 may be a topper and a mattress, respectively, and the vibration medium 21 may be a product of any of various types supported by the support member 22 as well as the topper. In addition, the contactless electrocardiogram measurement device 10 may directly transmit a vibration signal to the outside by measuring vibration generated from an object person, or calculate an electrocardiogram signal from the vibration signal and transmit the same to the outside. In this case, the contactless electrocardiogram measurement device 10 may further include a wireless communication means to enable its data communication with a user terminal 30 such as a smartphone 31 or a personal computer (PC) 32.

Referring to FIG. 3, the contactless electrocardiogram measurement device 10 may include a measurement unit 100 disposed between the vibration medium 21 and the support member 22, and a processor 200 calculating the electrocardiogram signal by using the vibration signal measured by the measurement unit 100. In this case, the measurement unit 100 and the processor 200 may perform data communication with each other by being connected to each other by wire through a cable C. Alternatively, the measurement unit 100 and the processor 200 may be wirelessly connected to each other by being equipped with a wireless communication means, respectively. Here, the measurement unit 100 may be disposed while being unexposed as the measurement unit is covered by the vibration medium 21, and the processor 200 may be disposed while being exposed to be manipulated. For example, the processor 200 may be disposed on a side surface of the support member 22.

Referring to FIG. 4, the measurement unit 100 may include a plate-shaped cover portion 110 interposed between the vibration medium 21 and the support member 22, and a sensor assembly 120 embedded in a cavity 22a of the support member 22. In this case, the sensor assembly 120 may be disposed at the center of the cover portion 110. Here, the cover portion 110 may have a diameter greater than a diameter of the cavity 22a of the support member 22, and thus be disposed while having an upper surface and a lower surface in contact with a lower surface of the vibration medium 21 and an upper surface of the support member 22, respectively. In addition, the cover portion 110 may have hardness greater than that of the vibration medium 21 to prevent the transmitted vibration from being attenuated. Accordingly, even if the vibration generated from a body of the object person is partially attenuated through the vibration medium 21, the cover portion 110 can separately suppress the vibration from being attenuated and more precisely detect a peak signal. In this case, the cover portion 110 may have an upper or lower thickness smaller than a front, rear, left or right diameter, and have a plate shape so that the object person does not feel direct foreign body sensation even when lying on the vibration medium 21. In addition, the sensor assembly 120 may be disposed in the cavity 22a of the support member 22, such that the object person may not feel direct foreign body sensation even when lying on the vibration medium 21.

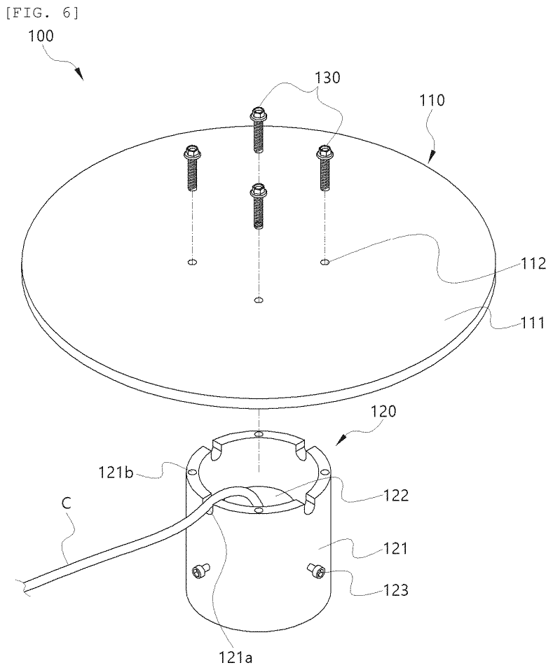

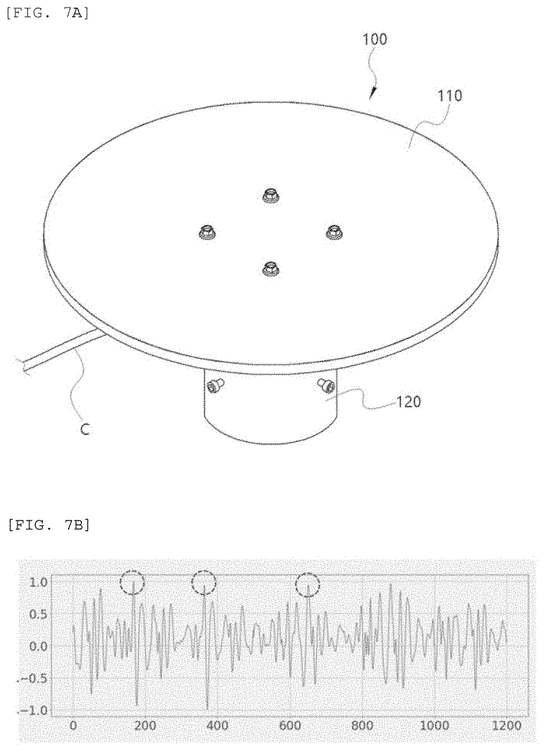

FIGS. 5 and 6 are related to the contactless electrocardiogram measurement device according to the present disclosure: FIG. 5 is a perspective view of the measurement unit; and FIG. 6 is an exploded perspective view of the measurement unit.

Referring to FIG. 5, the measurement unit 100 according to the present disclosure may include the plate-shaped cover portion 110 and the sensor assembly 120 coupled to one surface of the cover portion 110. The following description describes each component in more detail with reference to FIG. 6.

The cover portion 110 may include a plate-shaped cover body 111 having a front, rear, left or right diameter greater than its upper or lower thickness. In this case, the front, rear, left or right side of the cover body 111 may have any of various shapes such as a polygon or a circle, and the vibration can be transmitted more precisely over a large area when the cover body 111 has a disk shape. In this case, the cover portion 110 may further include a first screw hole 112 passing through the upper and lower surfaces of the cover body 111, and the measurement unit 100 may further include a fastening member 130 passing through the first screw hole 112 and coupling the cover portion 110 and the sensor assembly 120 to each other.

The sensor assembly 120 may include a cylinder-shaped case 121 having a hollow inside and an open top, and a vibration sensor 122 disposed in the hollow inside of the case 121. In addition, the vibration sensor 122 may measure the vibration signal transmitted through the cover body 111 and the case 121, or may be directly coupled to the cover body 111 to measure the vibration signal. In this case, the sensor assembly 120 may further include a fixing member 123 for fixing the vibration sensor 122 into the case 121.

The sensor assembly 120 may further include a seating groove 121a disposed in an upper surface of the case 121 to seat the cable C therein, and a second screw hole 121b disposed to face the first screw hole 112 and coupled with an end portion of the fastening member 130. Here, the cable C may be disposed to pass through a side surface of the case 121. Alternatively, as shown in FIG. 6, the upper surface of the case 121 and the lower surface of the cover body 111 may be in contact with each other and the case 121 may be partially recessed downward to insert the cable C thereinto. In addition, the case 121 may be disposed at the diameter center of the cover body 111 to transmit the vibration received in a larger area to the vibration sensor 122.

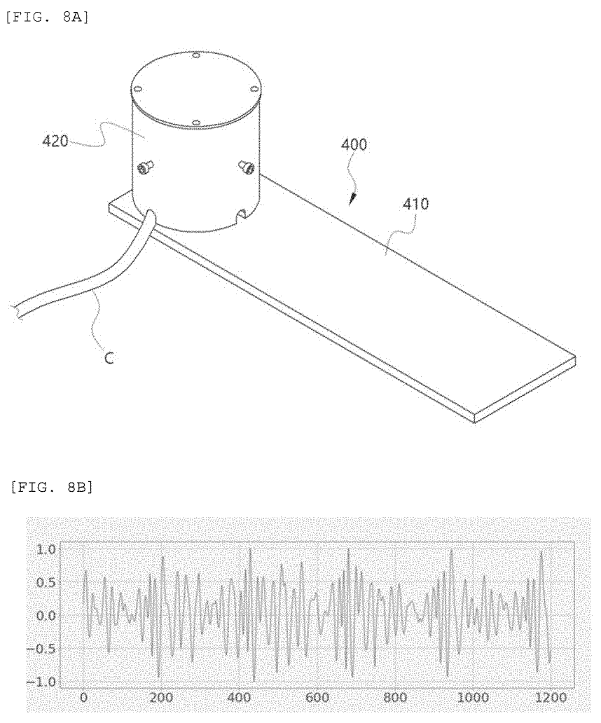

FIGS. 7A to 9B are views each comparing performance of the contactless electrocardiogram measurement device according to the present disclosure and that of a device having a different shape.

FIG. 7A is a perspective view of the measurement unit 100 according to the present disclosure, and FIG. 7B shows the vibration signal measured using the measurement unit 100, respectively. In addition, FIGS. 8A and 9A are perspective views of a steel use stainless (SUS) cantilever 400 and an artificial intelligence (AI) cantilever 500, respectively, and FIGS. 8B and 9B show the vibration signal measured by the SUS cantilever 400 and the AI cantilever 500, respectively.

Referring to FIG. 7A to 9B, the SUS cantilever 400 or the AI cantilever 500 may include a sensor unit 420 or 520 disposed on one side of a diaphragm 410 or 510 to detect the vibration in order to improve a sleep quality of the object person. In this case, a main peak may be often lost and a noise level may be higher as compared to the measurement unit 100, and distortion of the vibration signal may thus be relatively great. That is, the measurement unit 100 according to the present disclosure can significantly reduce the distortion of the vibration signal than the structure such as the SUS cantilever 400 or the AI cantilever 500, and thus obtain a more accurate vibration signal which can generate the artificial electrocardiogram signal.

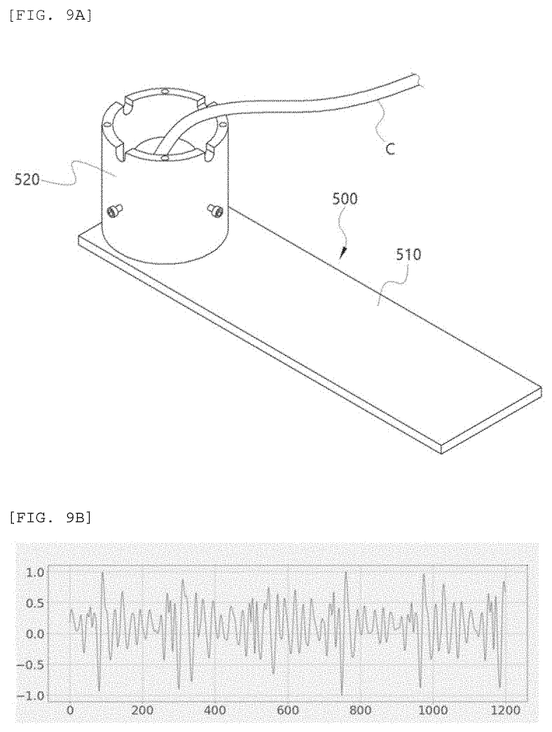

FIG. 10 is related to the contactless electrocardiogram measurement device according to the present disclosure, and is a cross-sectional view showing another example in which the measurement unit is disposed between the vibration medium and the support member according to the present disclosure.

Referring to FIG. 10, the measurement unit 100 may include the plate-shaped cover portion 110 interposed between the vibration medium 21 and the support member 22, and the sensor assembly 120 embedded in a cavity 21a of the vibration medium 21. In this case, the sensor assembly 120 may be disposed at the center of the cover portion 110. Here, the cover portion 110 may have a diameter greater than a diameter of the cavity 21a of the vibration medium 21, and thus be disposed while having the upper surface and the lower surface in contact with the lower surface of the vibration medium 21 and the upper surface of the support member 22, respectively. In addition, the cover portion 110 may have hardness greater than that of the vibration medium 21 to prevent the transmitted vibration from being attenuated. Accordingly, even if the vibration generated from the body of the object person is partially attenuated through the vibration medium 21, the cover portion 110 can separately suppress the vibration from being attenuated and more precisely detect the peak signal. In this case, the cover portion 110 may have the upper or lower thickness smaller than the front, rear, left or right diameter, and have the plate shape so that the object person does not feel the direct foreign body sensation even when lying on the vibration medium 21. In addition, the sensor assembly 120 may also be disposed in the cavity 21a of the vibration medium 21, such that the object person may not feel the direct foreign body sensation even when lying on the vibration medium 21.

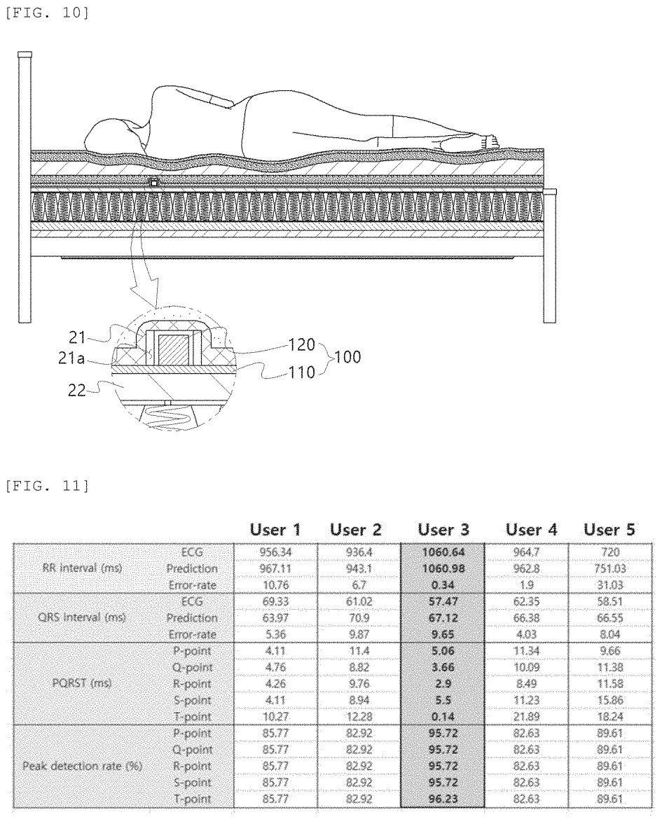

FIG. 11 is related to a method for calculating a contactless electrocardiogram signal by using a contactless electrocardiogram measurement device according to the present disclosure, and shows a table comparing actual measurement data and calculated data to which the method for calculating a contactless electrocardiogram signal according to the present disclosure is applied with each other.

Referring to FIG. 11, the method for calculating a contactless electrocardiogram signal according to the present disclosure includes: calculating a first electrocardiogram signal of an object person by using a contactless electrocardiogram measurement device; measuring a second electrocardiogram signal of the object person by using a contact electrocardiogram including electrodes; analyzing a correlation between the first electrocardiogram signal and the second electrocardiogram signal; and correcting data by reflecting the analyzed correlation in a calculation formula of the first electrocardiogram signal. That is, the contactless electrocardiogram measurement device may calculate the first electrocardiogram signal of the object person by using a vibration signal, and the time-synchronized contact electrocardiogram can measure the second electrocardiogram signal of the same object person to correct the calculation formula for calculating the first electrocardiogram signal by using the vibration signal. Here, the correlation between the first electrocardiogram signal and the second electrocardiogram signal may be trained using a deep learning algorithm, and the trained deep learning algorithm may be provided through a network or the like. A user provided with the trained deep learning algorithm may update algorithm of his/her contactless electrocardiogram measurement device in real time, thereby minimizing an error with actual measurement data. As shown in the table of FIG. 11, it can be seen that a case of user #3,to which the trained deep learning algorithm is reflected, has a lower error rate between the actual measurement data (or electrocardiogram (ECG)) and the calculated data (or prediction) and a higher peak detection rate as compared to a case of user #1, #2, #4 or #5.

As set forth above, the contactless electrocardiogram measurement device configured as described above according to the present disclosure may include the plate-shaped cover portion which is disposed between the vibration medium and the support member to more precisely measure the vibration generated from the object person, and the vibration sensor which is embedded in the vibration medium or the support member, thereby reducing the foreign body sensation felt by the object person. Accordingly, the contactless electrocardiogram measurement device according to the present disclosure may solve the conventional problems caused by the disturbed sleep of the object person, and may also be used in a place other than the specialized facility to be more widely used.

The contactless electrocardiogram measurement device according to the present disclosure may generate the electrocardiogram signal by using only the signal received from the vibration sensor without being attached to the body of the object person in sleep, and may thus derive the cardiovascular disease and precise health-related information of the object person by performing the high-quality sleep monitoring.

The contactless electrocardiogram measurement device according to the present disclosure may have higher reliability of the measurement result because the calculation formula is corrected using the data measured in the conventional electrocardiogram.

As described above, the present disclosure is described with reference to the specific matter such as the specific components, the specific exemplary embodiments and the drawings, which are provided only for assisting in the general understanding of the present disclosure. Therefore, the present disclosure is not limited to the exemplary embodiments. Various modifications and changes may be made by those skilled in the art to which the present disclosure pertains from this description.

Therefore, the spirit of the present disclosure should not be limited to the exemplary embodiments described above, and the claims and all of modifications equal or equivalent to the claims are intended to fall within the scope and spirit of the present disclosure.

What is claimed is:

1. A contactless electrocardiogram measurement device comprising a measurement unit disposed between a vibration medium and a support member to measure vibration generated from a body of an object person that is transmitted from the vibration medium,
wherein the measurement unit includes a plate-shaped cover portion interposed between the vibration medium and the support member, and a vibration sensor for detecting the vibration generated in the cover portion, and
the vibration sensor is embedded in the vibration medium or the support member.

2. The device of claim 1, wherein the vibration medium and the support member are a topper and a mattress, respectively.

3. The device of claim 1, further comprising a processor fixed on the support member and receiving data measured by the vibration sensor.

4. The device of claim 1, further comprising a case into which the vibration sensor is inserted and which has one side coupled to one surface of the cover portion, wherein the case has the one side open.

5. The device of claim 4, wherein the cover portion includes a plate-shaped cover body having a diameter greater than its thickness and a first screw hole passing through both surfaces of the cover body,
the case includes a second screw hole disposed in the one side thereof to face the first screw hole, and
the cover body and the case are screwed to each other.

6. The device of claim 4, wherein a seating groove into which a cable is able to be inserted is disposed in the one side of the case, and the cable is wired to the vibration sensor.

7. The device of claim 1, wherein the cover portion has a disk shape, and both the surfaces of the cover portion are in contact with the vibration medium and the support member, respectively.

8. The device of claim 7, wherein the vibration medium or the support member has a cavity disposed in its surface in contact with the cover portion to embed the vibration sensor therein, and an area of the cavity is smaller than an area of the cover portion.

9. The device of claim 7, wherein the case is disposed at a diameter center of the cover portion.

10. The device of claim 4, further comprising a fixing member for fixing the vibration sensor into the case.

11. The device of claim 1, wherein the cover portion has hardness greater than that of the vibration medium.

12. The device of claim 11, wherein the cover portion has a disk shape,
both surfaces of the cover portion are in contact with the vibration medium and the support member, respectively, and
a case into which the vibration sensor is inserted and which has one side coupled to one surface of the cover portion is disposed at a diameter center of the cover portion.

13. The device of claim 12, wherein the vibration medium or the support member has a cavity disposed in its surface in contact with the cover portion to embed the vibration sensor therein, and an area of the cavity is smaller than an area of the cover portion.

14. A method for calculating a contactless electrocardiogram signal, the method comprising:
calculating a first electrocardiogram signal of an object person by using the contactless electrocardiogram measurement device of claim 1;
measuring a second electrocardiogram signal of the object person by using a contact electrocardiogram including electrodes;
analyzing a correlation between the first electrocardiogram signal and the second electrocardiogram signal; and
correcting data by reflecting the analyzed correlation in a calculation formula of the first electrocardiogram signal.

15. The method of claim 14, wherein in the correcting, the correlation between the first electrocardiogram signal and the second electrocardiogram signal is trained using a deep learning algorithm.
